(19) United States
(12) Patent Application Publication
(10) Pub. No.: US 2013/0150862 A1
(43) Pub. Date: Jun. 13, 2013

(54) CUSTOMIZED PATIENT-SPECIFIC BONE CUTTING BLOCKS HAVING LOCATING FEATURES AND METHOD OF MAKING THE SAME

(71) Applicants: Luke J. Aram, Warsaw, IN (US); William Bugbee, Warsaw, IN (US); Charles A. Engh, Warsaw, IN (US); Joseph Moskal, Warsaw, IN (US); Mark Pagnano, Warsaw, IN (US); Michael Swank, Warsaw, IN (US); Bryan Rose, Warsaw, IN (US)

(72) Inventors: Luke J. Aram, Warsaw, IN (US); William Bugbee, Warsaw, IN (US); Charles A. Engh, Warsaw, IN (US); Joseph Moskal, Warsaw, IN (US); Mark Pagnano, Warsaw, IN (US); Michael Swank, Warsaw, IN (US); Bryan Rose, Warsaw, IN (US)

(21) Appl. No.: 13/580,260

(22) PCT Filed: Feb. 23, 2011
(86) PCT No.: PCT/US11/25907

§ 371 Date: Nov. 6, 2012

Related U.S. Application Data
(60) Provisional application No. 61/308,192, filed on Feb. 25, 2010.

(51) Int. Cl. A61B 17/17

(52) U.S. Cl. CPC A61B 17/1764
USPC 606/88; 29/592

(57) ABSTRACT

A number of orthopaedic surgical instruments are also disclosed. A method, apparatus, and system for fabricating such instruments are also disclosed.

This application claims priority under 35 U.S.C. §119(e) to U.S. Provisional Patent Application Ser. No. 61/308,192, entitled “Customized Patient-Specific Bone Cutting Blocks Having Locating Features and Method of Making the Same,” which was filed on Feb. 25, 2010 by Bryan Rose et al., the entirety of which is incorporated by reference.

### CROSS-REFERENCE TO RELATED U.S. PATENT APPLICATIONS

Cross-reference is made to co-pending U.S. Utility patent application Ser. Nos. 12/240,985; 12/240,990; 12/240,988; 12/240,992; 12/240,994; 12/240,996; 12/240,997; 12/240,998; 12/241,006; 12/241,002; 12/241,001; and 12/240,999. Each of these applications was filed on Sep. 29, 2008, and is assigned to the same assignee as the present application. Each of these applications is hereby incorporated by reference.

### TECHNICAL FIELD

The present disclosure relates generally to customized patient-specific orthopaedic surgical instruments and to methods, devices, and systems for fabricating and positioning such instruments.

### BACKGROUND

Joint arthroplasty is a well-known surgical procedure by which a diseased and/or damaged natural joint is replaced by a prosthetic joint. A typical knee prosthesis includes a tibial tray, a femoral component, a polymer insert or bearing positioned between the tibial tray and the femoral component, and, in some cases, a polymer patella button. To facilitate the replacement of the natural joint with the knee prosthesis, orthopaedic surgeons use a variety of orthopaedic surgical instruments such as, for example, cutting blocks, drill guides, milling guides, and other surgical instruments. Typically, the orthopaedic surgical instruments are generic with respect to the patient such that the same orthopaedic surgical instrument may be used on a number of different patients during similar orthopaedic surgical procedures.

### SUMMARY

According to one aspect, a method for designing a customized patient-specific bone cutting block for use in an orthopaedic surgical procedure to perform a bone cut on a patient's bone includes determining cartilage defect data indicative of the location, size, and shape of a cartilage defect present on an end of the patient's bone. The method also includes generating a reference contour based on the cartilage defect data, and, thereafter, creating a customized patient-specific negative contour of the customized patient-specific bone cutting block using the reference contour.

The reference contour may be generated based on a surface contour of a three-dimensional model of the patient's bone.

The cartilage defect data may be indicative of the location, size, and shape of a cartilage void. The customized patient-specific negative contour may include a protrusion that is sized, shaped, and positioned to be received into such a cartilage void when the customized patient-specific cutting block is secured to the patient's bone.

The cartilage defect data may be indicative of the location, size, and shape of a cartilage protrusion. The customized patient-specific negative contour may include a void that is sized, shaped, and positioned to receive such a cartilage protrusion when the customized patient-specific cutting block is secured to the patient's bone.

The cartilage defect data may include cartilage defect data associated with the distal end of the patient's femur, with such data being used to create a customized patient-specific negative contour of a customized patient-specific femoral cutting block.

The cartilage defect data may include cartilage defect data associated with the proximal end of the patient's tibia, with such data being used to create a customized patient-specific negative contour of a customized patient-specific tibial cutting block.

According to another aspect, a method for designing a customized patient-specific bone cutting block for use in an orthopaedic surgical procedure to perform a bone cut on a patient's bone includes determining cartilage defect data indicative of the location and size of a cartilage void present on an end of the patient's bone. The method also includes generating a reference contour based on the cartilage defect data, and, thereafter, creating a customized patient-specific negative contour of the customized patient-specific bone cutting block using the reference contour. The customized patient-specific negative contour includes a protrusion that is sized and positioned to be received into the cartilage void when the customized patient-specific cutting block is secured to the patient's bone.

The reference contour may be generated based on a surface contour of a three-dimensional model of the patient's bone.

The cartilage defect data may further include data indicative of the shape of the cartilage void.

The cartilage defect data may further include data indicative of the location, size, and shape of a cartilage protrusion present on the relevant end of the patient's bone. In such a case, the customized patient-specific negative contour may include a void that is sized, shaped, and positioned to receive the cartilage protrusion when the customized patient-specific cutting block is secured to the patient's bone.

The cartilage defect data may include cartilage defect data associated with the distal end of the patient's femur, with such data being used to create a customized patient-specific negative contour of a customized patient-specific femoral cutting block.

The cartilage defect data may include cartilage defect data associated with the proximal end of the patient's tibia, with such data being used to create a customized patient-specific negative contour of a customized patient-specific tibial cutting block.

According to another aspect, a customized patient-specific cutting block includes a bone-facing surface including a customized patient-specific negative contour configured to receive a portion of a patient's bone having a corresponding positive contour. The customized patient-specific negative contour includes a protrusion that is sized and positioned to be received into a cartilage void of corresponding size and position when the customized patient-specific cutting block is secured to the patient's bone.

The bone-facing surface may include a customized patient-specific negative contour configured to receive a portion of a patient's femur having a corresponding positive contour.

The bone-facing surface may include a customized patient-specific negative contour configured to receive a portion of a patient's tibia having a corresponding positive contour.

### BRIEF DESCRIPTION OF THE DRAWINGS

The detailed description particularly refers to the following figures, in which:

FIG. 1 is a simplified flow diagram of an algorithm for designing and fabricating a customized patient-specific orthopaedic surgical instrument;

FIG. 2 is a simplified flow diagram of a method for generating a model of a patient-specific orthopaedic instrument;

FIG. 3 is a simplified flow diagram of a method for scaling a reference contour;

FIGS. 4-6 are three-dimensional model's of a patient's tibia;

FIG. 7-9 are three-dimensional models of a patient's femur;

FIG. 10 is an anterior elevation an embodiment of a customized patient-specific orthopaedic surgical instrument;

FIG. 11 is a top plan view of the customized patient-specific orthopaedic surgical instrument of FIG. 10;

FIG. 12 is side elevation view of the customized patient-specific orthopaedic surgical instrument of FIG. 10;

FIG. 13 is a diagrammatic view of showing cartilage defects in the patient's distal femur;

FIG. 14 is a perspective view of the customized patient-specific orthopaedic surgical instrument of FIG. 10 showing the protrusions formed on the negative contour of the instrument that are received into the cartilage defects shown in FIG. 13;

FIG. 15 is an anterior elevation view of another embodiment of a customized patient-specific orthopaedic surgical instrument;

FIG. 16 is a top plan view of the customized patient-specific orthopaedic surgical instrument of FIG. 15;

FIG. 17 is side elevation view of the customized patient-specific orthopaedic surgical instrument of FIG. 15;

FIG. 18 is a diagrammatic view of showing cartilage defects in the patient's proximal tibia; and

FIG. 19 is a perspective view of the customized patient-specific orthopaedic surgical instrument of FIG. 15 showing the protrusions formed on the negative contour of the instrument that are received into the cartilage defects shown in FIG. 18.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout this disclosure in reference to the orthopaedic implants and instruments described herein, along with a patient's natural anatomy. Such terms have well-understood meanings in both the study of anatomy and the field of orthopaedics. Use of such anatomical reference terms in the specification and claims is intended to be consistent with their well-understood meanings unless noted otherwise.

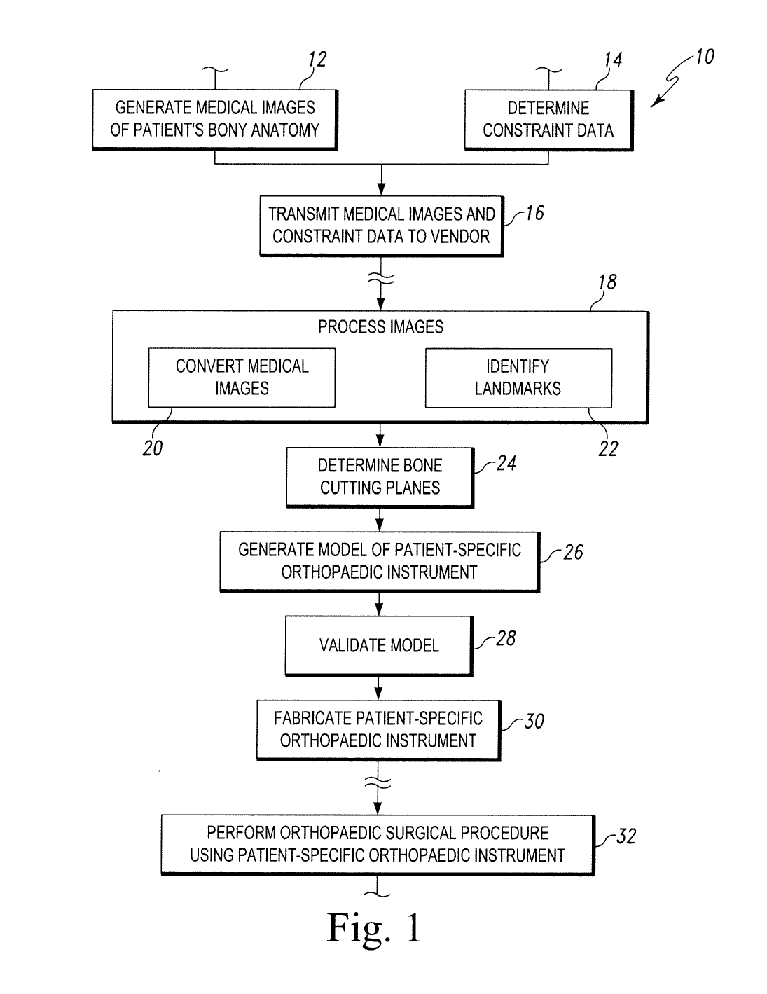

Referring to FIG. 1, an algorithm 10 for fabricating a customized patient-specific orthopaedic surgical instrument is illustrated. What is meant herein by the term “customized patient-specific orthopaedic surgical instrument” is a surgical tool for use by a surgeon in performing an orthopaedic surgical procedure that is intended, and configured, for use on a particular patient. As such, it should be appreciated that, as used herein, the term “customized patient-specific orthopaedic surgical instrument” is distinct from standard, non-patient specific orthopaedic surgical instruments that are intended for use on a variety of different patients. Additionally, it should be appreciated that, as used herein, the term “customized patient-specific orthopaedic surgical instrument” is distinct from orthopaedic prostheses, whether patient-specific or generic, which are surgically implanted in the body of the patient. Rather, customized patient-specific orthopaedic surgical instruments are used by an orthopaedic surgeon to assist in the implantation of orthopaedic prostheses.

In some embodiments, the customized patient-specific orthopaedic surgical instrument may be customized to the particular patient based on the location at which the instrument is to be coupled to one or more bones of the patient, such as the femur and/or tibia. For example, in some embodiments, the customized patient-specific orthopaedic surgical instrument may include a bone-contacting or facing surface having a negative contour that matches or substantially matches the contour of a portion of the relevant bone of the patient. As such, the customized patient-specific orthopaedic surgical instrument is configured to be coupled to the bone of a patient in a unique location and position with respect to the patient's bone. That is, the negative contour of the bone-contacting surface is configured to receive the matching contour surface of the portion of the patient's bone. As such, the orthopaedic surgeon's guesswork and/or intra-operative decision-making with respect to the placement of the orthopaedic surgical instrument are reduced. For example, the orthopaedic surgeon may not be required to locate landmarks of the patient's bone to facilitate the placement of the orthopaedic surgical instrument, which typically requires some amount of estimation on part of the surgeon. Rather, the orthopaedic surgeon may simply couple the customized patient-specific orthopaedic surgical instrument on the bone or bones of the patient in the unique location. When so coupled, the cutting plane, drilling holes, milling holes, and/or other guides are defined in the proper location relative to the bone and intended orthopaedic prosthesis. The customized patient-specific orthopaedic surgical instrument may be embodied as any type of orthopaedic surgical instrument such as, for example, a bone-cutting block, a drilling guide, a milling guide, or other type of orthopaedic surgical instrument configured to be coupled to a bone of a patient.

As shown in FIG. 1, the algorithm 10 includes process steps 12 and 14, in which an orthopaedic surgeon performs pre-operative planning of the orthopaedic surgical procedure to be performed on a patient. The process steps 12 and 14 may be performed in any order or contemporaneously with each other. In process step 12, a number of medical images of the relevant bony anatomy or joint of the patient are generated. To do so, the orthopaedic surgeon or other healthcare provider may operate an imaging system to generate the medical images. The medical images may be embodied as any number and type of medical images capable of being used to generate a three-dimensional rendered model of the patient's bony anatomy or relevant joint. For example, the medical images may be embodied as any number of computed tomography (CT) images, magnetic resonance imaging (MRI) images, or other three-dimensional medical images. Additionally or alternatively, as discussed in more detail below in regard to process step 18, the medical images may be embodied as a number of X-ray images or other two-dimensional images from which a three-dimensional rendered model of the patient's relevant bony anatomy may be generated. Additionally, in some embodiments, the medical image may be enhanced with a contrast agent designed to highlight the cartilage surface of the patient's knee joint.

In process step 14, the orthopaedic surgeon may determine any additional pre-operative constraint data. The constraint data may be based on the orthopaedic surgeon's preferences, preferences of the patient, anatomical aspects of the patient, guidelines established by the healthcare facility, or the like. For example, the constraint data may include the orthopaedic surgeon's preference for a metal-on-metal interface, amount of inclination for implantation, the thickness of the bone to resect, size range of the orthopaedic implant, and/or the like. In some embodiments, the orthopaedic surgeon's preferences are saved as a surgeon's profile, which may used as a default constraint values for further surgical plans.

In process step 16, the medical images and the constraint data, if any, are transmitted or otherwise provided to an orthopaedic surgical instrument vendor or manufacturer. The medical images and the constraint data may be transmitted to the vendor via electronic means such as a network or the like. After the vendor has received the medical images and the constraint data, the vendor processes the images in step 18. The orthopaedic surgical instrument vendor or manufacturer process the medical images to facilitate the determination of the bone cutting planes, implant sizing, and fabrication of the customized patient-specific orthopaedic surgical instrument as discussed in more detail below. For example, in process step 20 the vendor may convert or otherwise generate three-dimensional images from the medical images. For example, in embodiments wherein the medical images are embodied as a number of two-dimensional images, the vendor may use a suitable computer algorithm to generate one or more three-dimensional images form the number of two-dimensional images. Additionally, in some embodiments, the medical images may be generated based on an established standard such as the Digital Imaging and Communications in Medicine (DICOM) standard. In such embodiments, an edge-detection, thresholding, watershead, or shape-matching algorithm may be used to convert or reconstruct images to a format acceptable in a computer aided design application or other image processing application. Further, in some embodiments, an algorithm may be used to account for tissue such as cartilage not discernable in the generated medical images. In such embodiments, any three-dimensional model of the patient-specific instrument (see, e.g., process step 26 below) may be modified according to such algorithm to increase the fit and function of the instrument.

In process step 22, the vendor may process the medical images, and/or the converted/reconstructed images from process step 20, to determine a number of aspects related to the bony anatomy of the patient such as the anatomical axis of the patient's bones, the mechanical axis of the patient's bone, other axes and various landmarks, and/or other aspects of the patient's bony anatomy. To do so, the vendor may use any suitable algorithm to process the images.

In process step 24, the cutting planes of the patient's bone are determined. The planned cutting planes are determined based on the type, size, and position of the orthopaedic prosthesis to be used during the orthopaedic surgical procedure, on the process images such as specific landmarks identified in the images, and on the constraint data supplied by the orthopaedic surgeon in process steps 14 and 16. The type and/or size of the orthopaedic prosthesis may be determined based on the patient's anatomy and the constraint data. For example, the constraint data may dictate the type, make, model, size, or other characteristic of the orthopaedic prosthesis. The selection of the orthopaedic prosthesis may also be modified based on the medical images such that an orthopaedic prosthesis that is usable with the bony anatomy of the patient and that matches the constraint data or preferences of the orthopaedic surgeon is selected.

In addition to the type and size of the orthopaedic prosthesis, the planned location and position of the orthopaedic prosthesis relative to the patient's bony anatomy is determined. To do so, a digital template of the selected orthopaedic prosthesis may be overlaid onto one or more of the processed medical images. The vendor may use any suitable algorithm to determine a recommended location and orientation of the orthopaedic prosthesis (i.e., the digital template) with respect to the patient's bone based on the processed medical images (e.g., landmarks of the patient's bone defined in the images) and/or the constraint data. Additionally, any one or more other aspects of the patient's bony anatomy may be used to determine the proper positioning of the digital template.

In some embodiments, the digital template along with surgical alignment parameters may be presented to the orthopaedic surgeon for approval. The approval document may include the implant's rotation with respect to bony landmarks such as the femoral epicondyle, posterior condyles, sulcus groove (Whiteside's line), and the mechanical axis as defined by the hip, knee, and/or ankle centers.

The planned cutting planes for the patient's bone(s) may then be determined based on the determined size, location, and orientation of the orthopaedic prosthesis. In addition, other aspects of the patient's bony anatomy, as determined in process step 22, may be used to determine or adjust the planned cutting planes. For example, the determined mechanical axis, landmarks, and/or other determined aspects of the relevant bones of the patient may be used to determine the planned cutting planes.

In process step 26, a model of the customized patient-specific orthopaedic surgical instrument is generated. In some embodiments, the model is embodied as a three-dimensional rendering of the customized patient-specific orthopaedic surgical instrument. In other embodiments, the model may be embodied as a mock-up or fast prototype of the customized patient-specific orthopaedic surgical instrument. The particular type of orthopaedic surgical instrument to be modeled and fabricated may be determined based on the orthopaedic surgical procedure to be performed, the constraint data, and/or the type of orthopaedic prosthesis to be implanted in the patient. As such, the customized patient-specific orthopaedic surgical instrument may be embodied as any type of orthopaedic surgical instrument for use in the performance of an orthopaedic surgical procedure. For example, the orthopaedic surgical instrument may be embodied as a bone-cutting block, a drilling guide, a milling guide, and/or any other type of orthopaedic surgical tool or instrument.

The particular shape of the customized patient-specific orthopaedic surgical instrument is determined based on the planned location of the orthopaedic surgical instrument relative to the patient's bony anatomy. The location of the customized patient-specific orthopaedic surgical instrument with respect to the patient's bony anatomy is determined based on the type and determined location of the orthopaedic prosthesis to be used during the orthopaedic surgical procedure. That is, the planned location of the customized patient-specific orthopaedic surgical instrument relative to the patient's bony anatomy may be selected based on, in part, the planned cutting planes of the patient's bone(s) as determined in step 24. For example, in embodiments wherein the customized patient-specific orthopaedic surgical instrument is embodied as a bone-cutting block, the location of the orthopaedic surgical instrument is selected such that the cutting guide of the bone-cutting block matches one or more of the planned cutting planes determined in process step 24. Additionally, the planned location of the orthopaedic surgical instrument may be based on the identified landmarks of the patient's bone identified in process step 22.

In some embodiments, the particular shape or configuration of the customized patient-specific orthopaedic surgical instrument may be determined based on the planned location of the instrument relative to the patient's bony anatomy. That is, the customized patient-specific orthopaedic surgical instrument may include a bone-contacting surface having a negative contour that matches the contour of a portion of the bony anatomy of the patient such that the orthopaedic surgical instrument may be coupled to the bony anatomy of the patient in a unique location, which corresponds to the pre-planned location for the instrument. When the orthopaedic surgical instrument is coupled to the patient's bony anatomy in the unique location, one or more guides (e.g., cutting or drilling guide) of the orthopaedic surgical instrument may be aligned to one or more of the bone cutting plane(s) as discussed above.

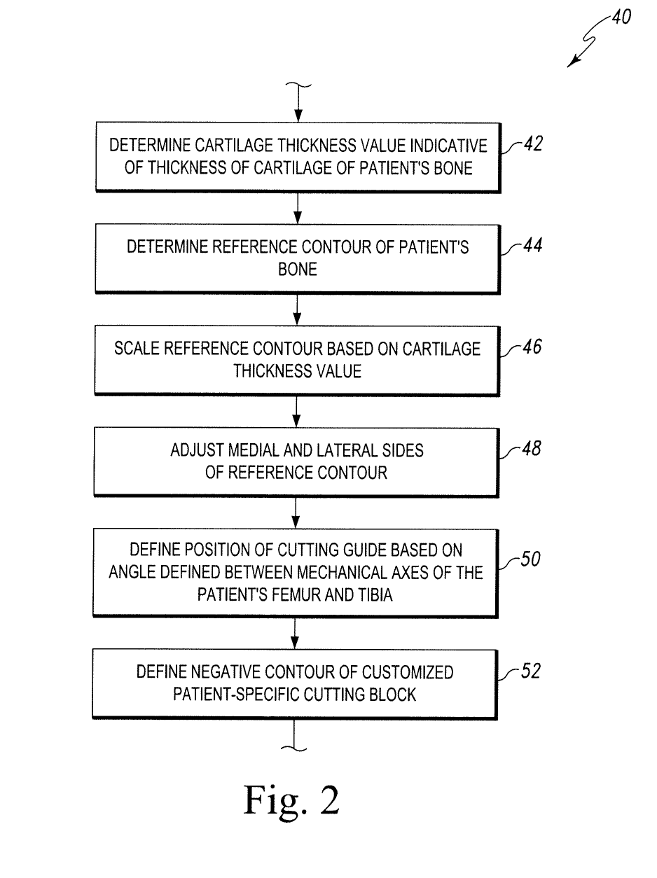

One illustrative embodiment of a method 40 for generating a model, such as a computer model, of a patient-specific orthopaedic instrument is illustrated in FIGS. 2 through 9. The method 40 begins with a step 42 in which a cartilage thickness value is determined. The cartilage thickness value is indicative of the average thickness of the cartilage of the patient's bone. As such, in one embodiment, the cartilage thickness value is equal to the average thickness of cartilage for an individual having similar characteristics as the patient. For example, the cartilage thickness value may be equal to the average thickness value of individuals of the same gender as the patient, the same age as the patient, having the same activity level of the patient, and/or the like. In other embodiments, the cartilage thickness value is determined based on one or more medical images of the patient's bone, such as those images transmitted in process step 16.

In step 44, a reference contour of the patient's relevant bone is determined. The reference contour is based on the surface contour of a three-dimensional model of the patient's relevant bone, such as the three-dimensional model generated in step 20. Initially the reference contour is identical to a region (i.e. the region of interest such as the distal end of the patient's femur or the proximal end of the patient's tibia) of the patient's bone. That is, in some embodiments, the reference contour is juxtaposed on the surface contour of the region of the patient's bone.

Subsequently, in step 46, the reference contour is scaled to compensate for the cartilage thickness value determined in step 42. To do so, in one embodiment, the scale of the reference contour is increased based on the cartilage thickness value. For example, the scale of the reference contour may be increased by an amount equal to or determined from the cartilage thickness value. However, in other embodiments, the reference contour may be scaled using other techniques designed to scale the reference contour to a size at which the reference contour is compensated for the thickness of the cartilage on the patient's bone.

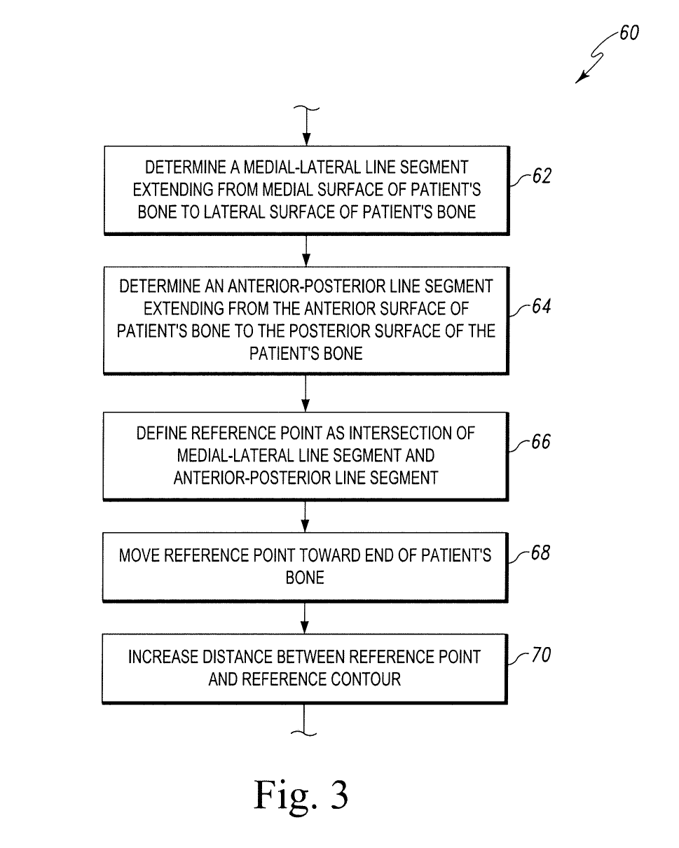

For example, in one particular embodiment, the reference contour is scaled by increasing the distance between a fixed reference point and a point lying on, and defining in part, the reference contour. To do so, in one embodiment, a method 60 for scaling a reference contour as illustrated in FIG. 3 may be used. The method 60 begins with step 62 in which a medial/lateral line segment is established on the three-dimensional model of the patient's relevant bone. The medial/lateral line segment is defined or otherwise selected so as to extend from a point lying on the medial surface of the patient's bone to a point lying on lateral surface of the patient's bone. The medial surface point and the lateral surface point may be selected so as to define the substantially maximum local medial/lateral width of the patient's bone in some embodiments.

In step 64, an anterior/posterior line segment is established on the three-dimensional model of the patient's relevant bone. The anterior/posterior line segment is defined or otherwise selected so as to extend from a point lying on the anterior surface of the patient's bone to a point lying on posterior surface of the patient's bone. The anterior surface point and the posterior surface point may be selected so as to define the substantially maximum local anterior/posterior width of the patient's bone in some embodiments.

The reference point from which the reference contour will be scaled is defined in step 66 as the intersection point of the medial/lateral line segment and anterior/posterior line segment. As such, it should be appreciated that the medial surface point, the lateral surface point, the anterior surface point, and the posterior surface point lie on the same plane. After the reference point is initially established in step 66, the reference point is moved or otherwise translated toward an end of the patient's bone. For example, in embodiments wherein the patient's bone is embodied as a femur, the reference point is moved inferiorly toward the distal end of the patient's femur. Conversely, in embodiments when the patient's bone is embodied as a tibia, the reference point is moved superiorly toward the proximal end of the patient's tibia. In one embodiment, the reference point is moved a distance equal to about half the length of the anterior/posterior line segment as determined in step 64. However, in other embodiments, the reference point may be moved other distances sufficient to compensate the reference contour for thickness of the cartilage present on the patient's bone.

Once the location of the reference point has been determined in step 68, the distance between the reference point and each point lying on, and defining in part, the reference contour is increased in step 70. To do so, in one particular embodiment, each point of the reference contour is moved a distance away from the reference point based on a percentage value of the original distance defined between the reference point and the particular point on the reference contour. For example, in one embodiment, each point lying on, and defining in part, the reference contour is moved away from the reference point in by a distance equal to a percentage value of the original distance between the reference point and the particular point. In one embodiment, the percentage value is in the range of about 5 percent to about thirty percent. In one particular embodiment, the percentage value is about ten percent.

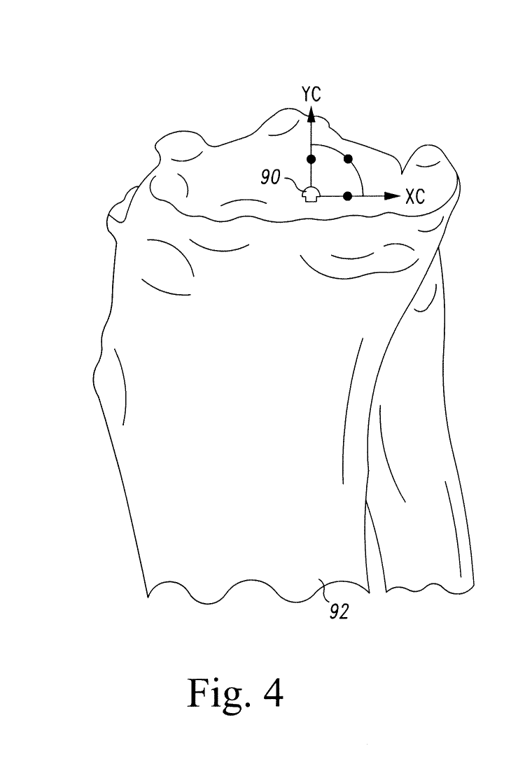

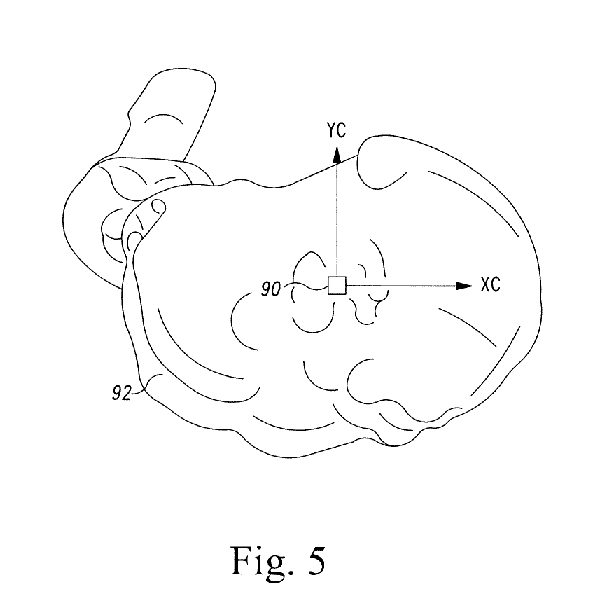

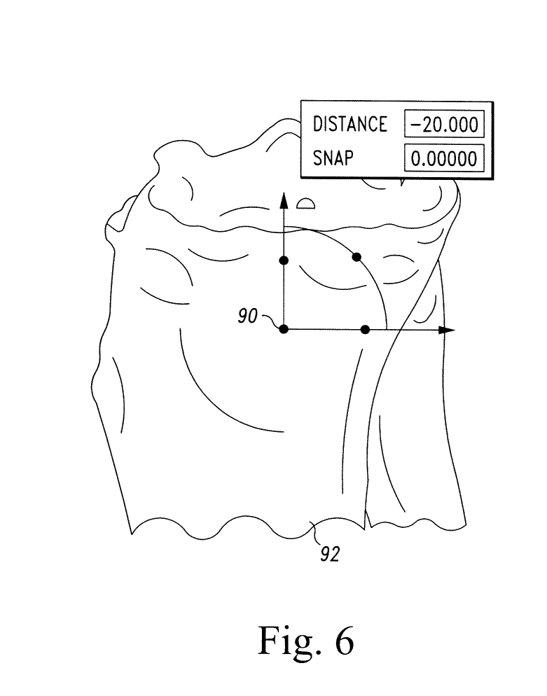

Referring now to FIGS. 4-9, in another embodiment, the reference contour is scaled by manually selecting a local “high” point on the surface contour of the three-dimensional image of the patient's bone. For example, in embodiments wherein the relevant patient's bone is embodied as a tibia as illustrated in FIGS. 4-6, the reference point 90 is initially located on the tibial plateau high point of the tibial model 92. Either side of the tibial plateau may be used. Once the reference point 90 is initially established on the tibial plateau high point, the reference point 90 is translated to the approximate center of the plateau as illustrated in FIG. 5 such that the Z-axis defining the reference point is parallel to the mechanical axis of the tibial model 92. Subsequently, as illustrated in FIG. 6, the reference point is moved in the distal direction by a predetermined amount. In one particular embodiment, the reference point is moved is the distal direction by about 20 millimeters, but other distances may be used in other embodiments. For example, the distance over which the reference point is moved may be based on the cartilage thickness value in some embodiments.

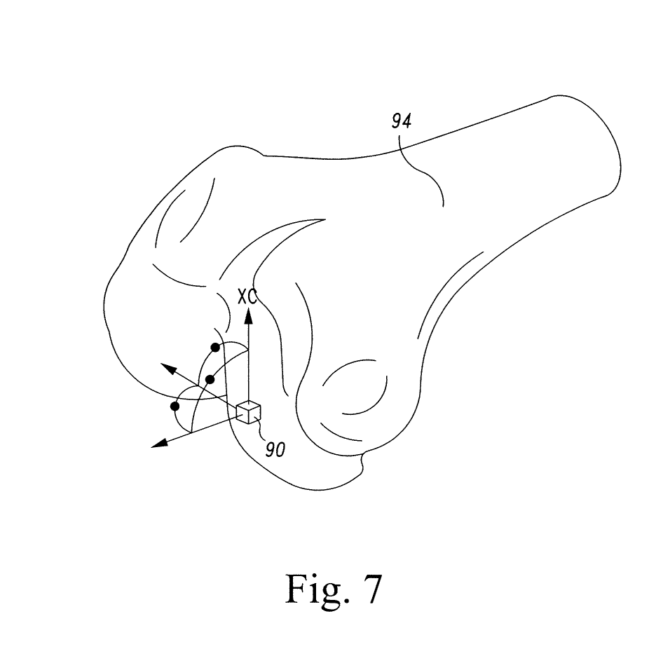

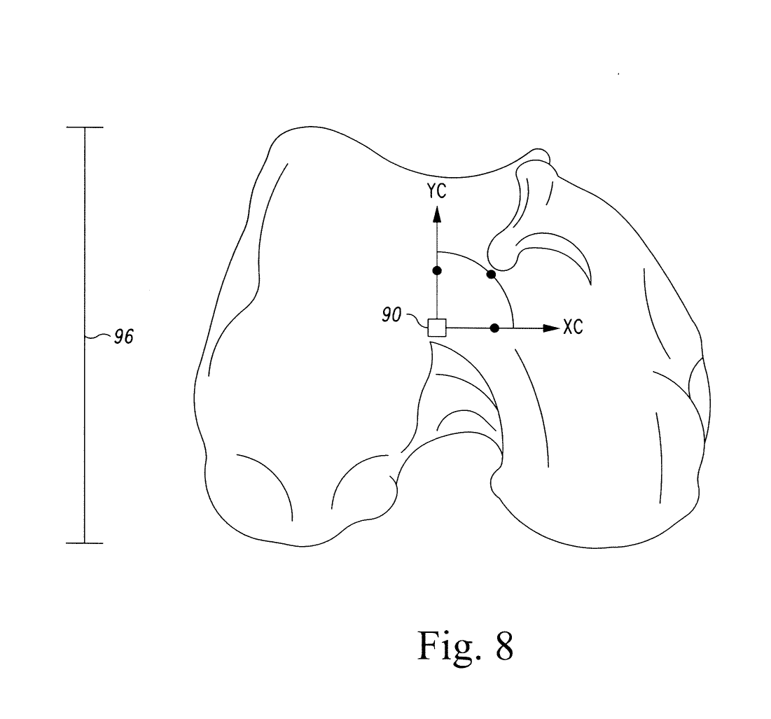

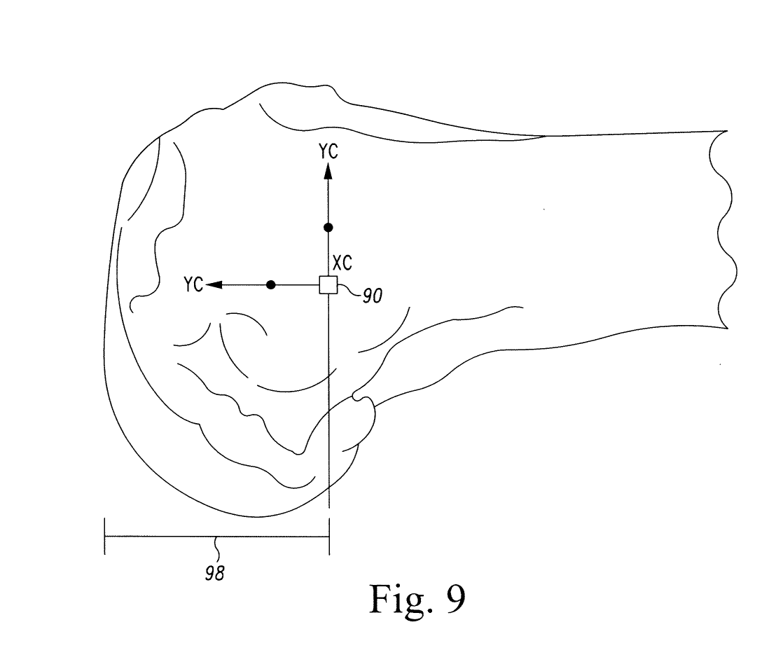

Conversely, in embodiments wherein the relevant patient's bone is embodied as a femur as illustrated in FIGS. 7-9, the reference point 90 is initially located on the most distal point of the distal end of the femoral model 94. Either condyle of the femoral model 94 may be used in various embodiments. Once the reference point 90 is initially established on the most distal point, the reference point 90 is translated to the approximate center of the distal end of the femoral model 94 as illustrated in FIG. 8 such that the Z-axis defining the reference point 90 is parallel to the mechanical axis of the femoral model 92. The anterior-posterior width 96 of the distal end of the femoral model 94 is also determined. Subsequently, as illustrated in FIG. 9, the reference point is moved or otherwise translated in the proximal or superior direction by a distance 98. In one particular embodiment, the reference point is moved in the distal or superior direction by a distance 98 equal to about half the distance 96. As such, it should be appreciated that one of a number of different techniques may be used to define the location of the reference point based on, for example, the type of bone.

Referring now back to FIG. 2, once the reference contour has been scaled in step 46, the medial/lateral sides of the reference contour are adjusted in step 48. To do so, in one embodiment, the distance between the reference point and each point lying on, and defining in part, the medial side and lateral side of the reference contour is decreased. For example, in some embodiments, the distance between the reference point and the points on the medial and lateral sides of the scaled reference contour are decreased to the original distance between such points. As such, it should be appreciated that the reference contour is offset or otherwise enlarged with respect to the anterior side of the patient's bone and substantially matches or is otherwise not scaled with respect to the medial and lateral sides of the patient's bone.

The reference contour may also be adjusted in step 48 for areas of the patient's bone having a reduced thickness of cartilage. Such areas of reduced cartilage thickness may be determined based on the existence of bone-on-bone contact as identified in a medical image, simulation, or the like. Additionally, information indicative of such areas may be provided by the orthopaedic surgeon based on his/her expertise. If one or more areas of reduced cartilage thickness are identified, the reference contour corresponding to such areas of the patient's bone is reduced (i.e., scaled back or down).

Additionally, in some embodiments, one or more osteophytes on the patient's bone may be identified; and the reference contour may be compensated for such presence of the osteophytes. By compensating for such osteophytes, the reference contour more closely matches the surface contour of the patient's bone. Further, in some embodiments, a distal end (in embodiments wherein the patient's bone is embodied as a tibia) or a proximal end (in embodiments wherein the patient's bone is embodied as a femur) of the reference contour may be adjusted to increase the conformity of the reference contour to the surface contour of the bone. For example, in embodiments wherein the patient's bone is a femur, the superior end of the scaled reference contour may be reduced or otherwise moved closer to the surface contour of the patient's femur in the region located superiorly to a cartilage demarcation line defined on the patient's femur. Conversely, in embodiments wherein the patient's bone is embodied as a tibia, an inferior end of the scaled reference contour may be reduced or otherwise moved closer to the surface contour of the patient's tibia in the region located inferiorly to a cartilage demarcation line of the patient's tibia. As such, it should be appreciated that the scaled reference contour is initially enlarged to compensate for the thickness of the patient's cartilage on the patient's bone. Portions of the scaled reference contour are then reduced or otherwise moved back to original positions and/or toward the reference point in those areas where cartilage is lacking, reduced, or otherwise not present.

Once the reference contour has been scaled and adjusted in steps 46 and 48, the position of the cutting guide is defined in step 50. In particular, the position of the cutting guide is defined based on an angle defined between a mechanical axis of the patient's femur and a mechanical axis of the patient's tibia. The angle may be determined by establishing a line segment or ray originating from the proximal end of the patient's femur to the distal end of the patient's femur and defining a second line segment or ray extending from the patient's ankle through the proximal end of the patient's tibia. The angle defined by these two line segments/rays is equal to the angle defined between the mechanical axis of the patient's femur and tibia. The position of the bone cutting guide is then determined based on the angle between the mechanical axes of the patient's femur and tibia. It should be appreciated that the position of the cutting guide defines the position and orientation of the cutting plane of the customized patient-specific cutting block. Subsequently, in step 52, a negative contour of the customized patient-specific cutting block is defined based on the scaled and adjusted reference contour and the angle defined between the mechanical axis of the femur and tibia.

Referring back to FIG. 1, after the model of the customized patient-specific orthopaedic surgical instrument has been generated in process step 26, the model is validated in process step 28. The model may be validated by, for example, analyzing the rendered model while coupled to the three-dimensional model of the patient's anatomy to verify the correlation of cutting guides and planes, drilling guides and planned drill points, and/or the like. Additionally, the model may be validated by transmitting or otherwise providing the model generated in step 26 to the orthopaedic surgeon for review. For example, in embodiments wherein the model is a three-dimensional rendered model, the model along with the three-dimensional images of the patient's relevant bone(s) may be transmitted to the surgeon for review. In embodiments wherein the model is a physical prototype, the model may be shipped to the orthopaedic surgeon for validation.

After the model has been validated in process step 28, the customized patient-specific orthopaedic surgical instrument is fabricated in process step 30. The customized patient-specific orthopaedic surgical instrument may be fabricated using any suitable fabrication device and method. Additionally, the customized patient-specific orthopaedic instrument may be formed from any suitable material such as a metallic material, a plastic material, or combination thereof depending on, for example, the intended use of the instrument. The fabricated customized patient-specific orthopaedic instrument is subsequently shipped or otherwise provided to the orthopaedic surgeon. The surgeon performs the orthopaedic surgical procedure in process step 32 using the customized patient-specific orthopaedic surgical instrument. As discussed above, because the orthopaedic surgeon does not need to determine the proper location of the orthopaedic surgical instrument intra-operatively, which typically requires some amount of estimation on part of the surgeon, the guesswork and/or intra-operative decision-making on part of the orthopaedic surgeon is reduced.

As described above, the reference contour may also be adjusted in step 48 for areas of the patient's bone having a reduced thickness of cartilage. Such areas of reduced cartilage thickness may be determined based on the existence of bone-on-bone contact as identified in a medical image, simulation, or the like. Additionally, information indicative of such areas may be provided by the orthopaedic surgeon based on his/her expertise.

Cartilage defect data may also be directly obtained from medical images. Such defect data may include the size, shape, and position of a cartilage defect, such as a cartilage void. Such defect data may be obtained by, for example and amongst other ways, analyzing one or more of: joint space measurements from standing x-rays, varus-valgus alignment measurements from standing x-rays, the position of bones in CT scan, any cartilage visible in CT scan, along with patient size, age, gender, and/or disease state.

Armed with this data, the reference contour may be adjusted based on the cartilage defect data. Namely, once the size, shape, and position of the cartilage voids is known, the reference contour may be altered to generate a protrusion that is the negative of the void. Specifically, a protrusion is created that is sized, shaped, and positioned to fit in the cartilage void when the instrument is secured to the patient's bone. In such a way, the protrusion functions as a locating feature to better position the customized patient-specific orthopaedic surgical instrument to the patient's bone.

It should be appreciated that if a cartilage or bony protrusion is pre-operatively discovered, the opposite approach may be used. That is, the reference contour may be altered to include a void that is sized, shaped, and located to match the size, shape, and location of the cartilage or bony protrusion on the patient's bone.

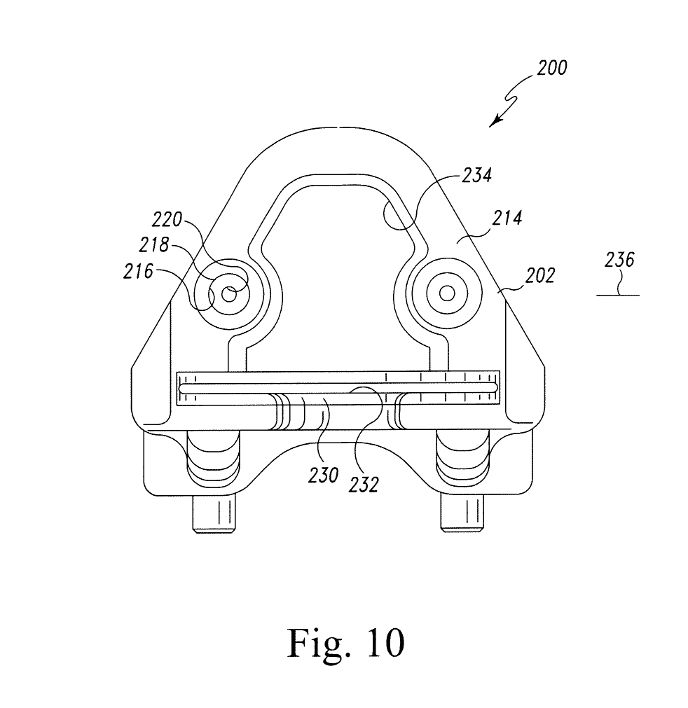

Referring now to FIGS. 10-14, in one embodiment, the customized patient-specific orthopaedic surgical instrument may be embodied as a femoral cutting block 200. The cutting block 200 is configured to be coupled to a femur of a patient. The cutting block 200 includes a body 202 configured to be coupled to the anterior side of the patient's femur and two arms or tabs 204, 206, which extend away from the body 202 in a posteriorly direction. The tabs 204, 206 are configured to wrap around a distal end of the femur as discussed in more detail below. Each of the tabs 204, 206 includes an inwardly-curving or otherwise superiorly extending lip 208, 210, respectively, which references the posterior condyles of the femur. The cutting block 200 may be formed from any suitable material. For example, the cutting block 200 may be formed from a material such as a plastic or resin material. In one particular embodiment, the cutting block 200 is formed from Vero resin using a rapid prototype fabrication process. However, the cutting block 200 may be formed from other materials in other embodiments. For example, in another particular embodiment, the cutting block 200 is formed from a polyimide thermoplastic resin, such as a Ultem resin, which is commercially available from Saudi Basic Industries Corporation Innovative Plastics of Riyhadh, Saudi Arabia.

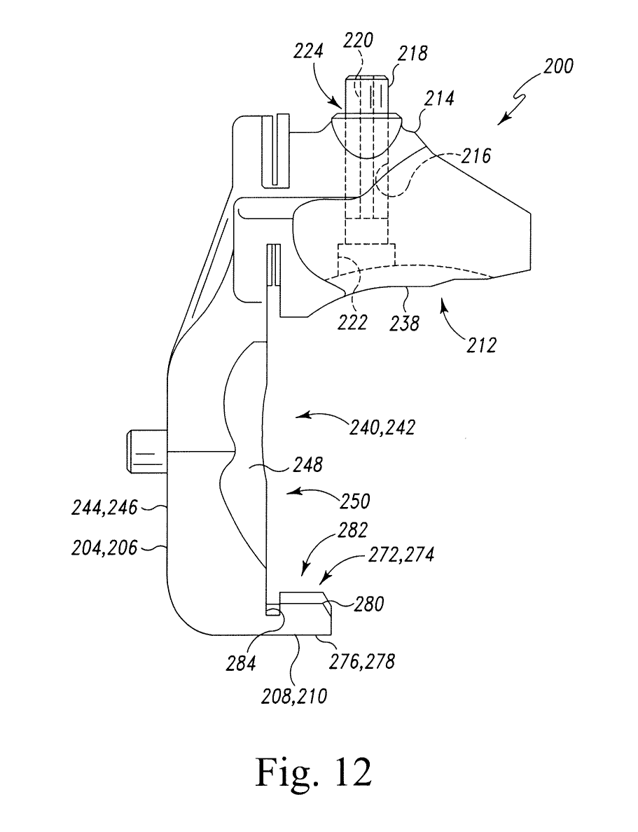

The body 202 includes a bone-contacting or bone-facing surface 212 and an outer surface 214 opposite the bone-facing surface 212. The outer surface 214 includes a number of guide holes or passageways 216 defined therethrough. A guide pin bushing 218 is received in each guide hole 216. The guide pin bushings 218 include an internal passageway 220 sized to receive a respective guide pin to secure the block 200 to the patient's femur. As shown in FIG. 12, the guide passageways 216 extends from the outer surface 214 to the bone-facing surface 212 and is counterbored on the bone-facing surface 212. That is, the passageway 216 has an opening 222 on the bone-facing surface 212 having a diameter greater than the diameter of an opening 224 on the outer surface 214

The cutting block 200 includes a cutting guide 230 secured to the body 202. In one particular embodiment, the cutting guide 230 is overmolded to the body 202. The cutting guide 230 includes a cutting guide slot 232. The cutting guide 230 may be formed from the same material as the body 202 or from a different material. In one particular embodiment, the cutting guide 230 is formed from a metallic material such as stainless steel. The body 202 also includes a window or opening 234 defined therethough. The opening 234 allows a surgeon to visualize the positioning of the block 200 on the patient's femur by viewing portions of the femur through the opening 234. Additionally, the opening 234 may reduce the amount of air pockets or other perfections created during the fabrication of the block 200. In the illustrative embodiment, the opening 234 extends from the cutting guide 200 to a point more superior than the superior-most point 236 of the guide pin bushings 218. However, in other embodiments, the cutting block 200 may include windows or openings formed in the body 202 having other shapes and sizes.

The bone-facing surface 212 of the body 202 includes a negative contour 238 configured to receive a portion of the anterior side of the patient's femur having a corresponding contour. As discussed above, the customized patient-specific negative contour 238 of the bone-contacting surface 212 allows the positioning of the cutting block 200 on the patient's femur in a unique pre-determined location and orientation.

The tabs 204, 206 include a bone-contacting or bone-facing surface 240, 242, respectively, and an outer surface 244, 246, respectively, opposite the bone-facing surface 240, 242. The bone-facing surface 240 of the tab 204 includes a negative contour 248 configured to receive a portion of the distal side of the patient's femur having a respective corresponding contour. Similarly, the bone-facing surface 242 of the tab 206 includes a negative contour 250 configured to receive a portion of the distal side of the patient's femur having a respective corresponding contour.

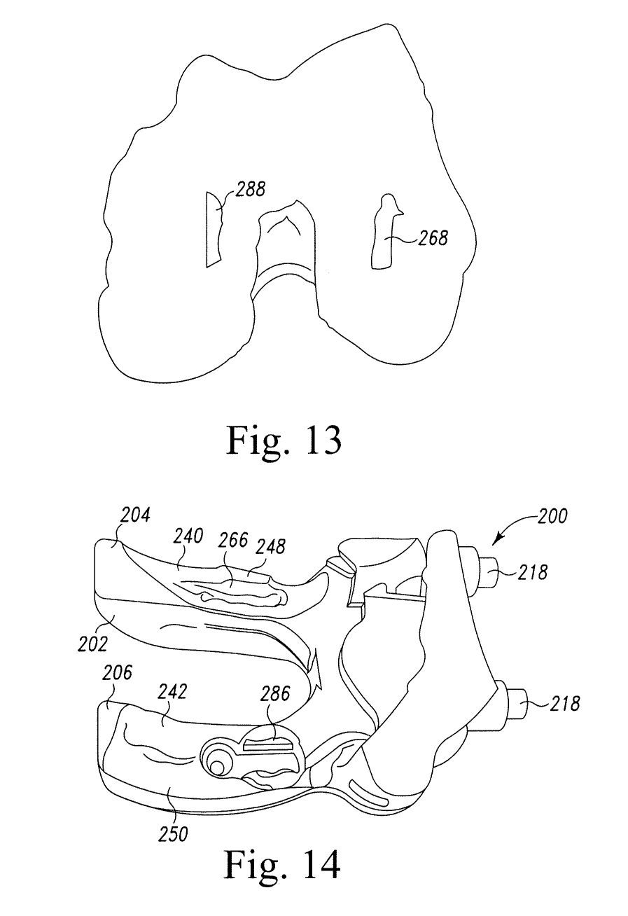

As can be seen in FIGS. 13 and 14, each of the negative contours 248, 250 of the tabs 204, 206, respectively includes a protrusion that corresponds to a cartilage void located on the distal end of the patient's femur. In particular, the negative contour 248 of the tab 204 includes a protrusion 266 that is sized, shaped, and positioned to be snuggly received into a cartilage defect 268 located on the patient's distal femur. The size, shape, and position of the cartilage defect 268 was pre-operatively determined as described above and the corresponding data was utilized in the fabrication of the cutting block 200. Similarly, the negative contour 250 of the tab 206 includes a protrusion 286 that is sized, shaped, and positioned to be snuggly received into a cartilage defect 288 located on the patient's distal femur. Like the cartilage defect 268, the size, shape, and position of the cartilage defect 288 was pre-operatively determined as described above and the corresponding data was utilized in the fabrication of the cutting block 200.

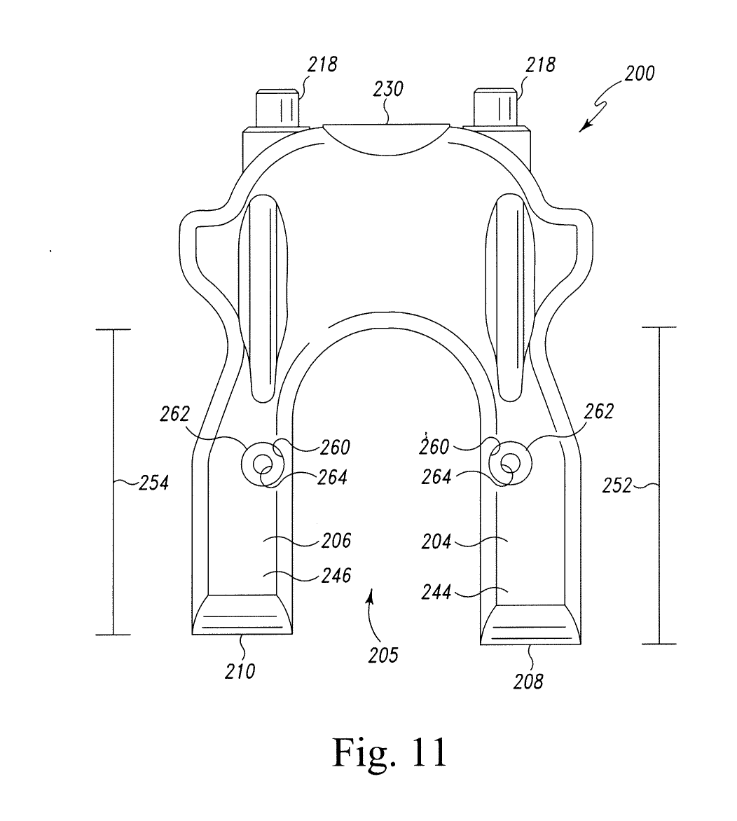

As discussed above, the arms or tabs 204, 206 extend posteriorly from the body 200 to define a U-shaped opening 205 therebetween. The tabs 204, 206 may extend from the body 202 the same distance or a different distance. For example, as shown in FIG. 11, the tab 204 extends from the body 202 a distance 252 and the tab 206 extends from the body 202 a distance 254, which is less than the distance 252. Each of the tabs 204, 206 includes a respective guide hole or passageway 260 defined therethrough. A guide pin bushing 262 is received in each guide hole 260. The guide pin bushings 262 include an internal passageway 264 sized to receive a respective guide pin to further secure the block 200 to the patient's femur. Similar to the guide passageways 216, the guide passageways 260 may be counterbored on the bone-facing surface 240, 242 of the tabs 204, 206.

The lips 208, 210 of the tabs 204, 206 also include a bone-contacting or bone-facing surface 272, 274, respectively, and an outer surface 276, 278, respectively, opposite the bone-facing surface 272, 274. The bone-facing surface 272 of the lip 208 includes a negative contour 280 configured to receive a portion of the posterior side of the patient's femur having a respective corresponding contour. Similarly, the bone-facing surface 274 of the lip 210 includes a negative contour 282 configured to receive a portion of the posterior side of the patient's femur having a respective corresponding contour. Each the lips 208, 210 include a lateral slot 284 that forms a saw relief slot and is configured to provide an amount of clearance for the bone saw blade used to remove a portion of the patient's bone. That is, during the performance of the orthopaedic surgical procedure, a distal end of the bone saw blade may be received in the slot 284.

In addition, in some embodiments, the negative contours 238, 248, 250, 280, 282 of the bone-contacting surfaces 212, 240, 242, 272, 274 of the cutting block 200 may or may not match the remaining corresponding contour surface of the patient's bone. That is, as discussed above, the negative contours 238, 248, 250, 280, 282 may be scaled or otherwise resized (e.g., enlarged) to compensate for the patient's cartilage or lack thereof.

In use, the femoral cutting block 200 is coupled to the distal end of the patient's femur. Again, because the bone-contacting surfaces 212, 240, 242, 272, 274 of the cutting block 200 include the negative contours 238, 248, 250, 280, 282, the block 200 may be coupled to the patient's femur in a pre-planned, unique position. When so coupled, the tabs 204, 206 wrap around the distal end of the patient's femur and the lips 208, 210 of the tabs 204, 206 wrap around the posterior side of the patient's femur. Additionally, when the block 200 is coupled to the patient's femur, a portion of the anterior side of the femur is received in the negative contour 238 of the body 202, a portion of the distal side of the patient's femur is received in the negative contours 248, 250 of the tabs 204, 206, and a portion of the posterior side of the femur is received in the negative contours 280, 282 of the lips 208, 210. As such, the anterior, distal, and posterior surfaces of the patient femur are referenced by the femoral cutting block 200. Moreover, when the distal end of the patient's femur is received into the negative contours 248, 250 of the tabs 204, 206, the protrusion 266 formed on the tab 204 is snuggly received into the cartilage defect 268, and the protrusion 268 formed on the tab 206 is snuggly received into the cartilage defect 288, thereby facilitating placement of the cutting block 200 in the desired, pre-operatively determined location on the patient's femur.

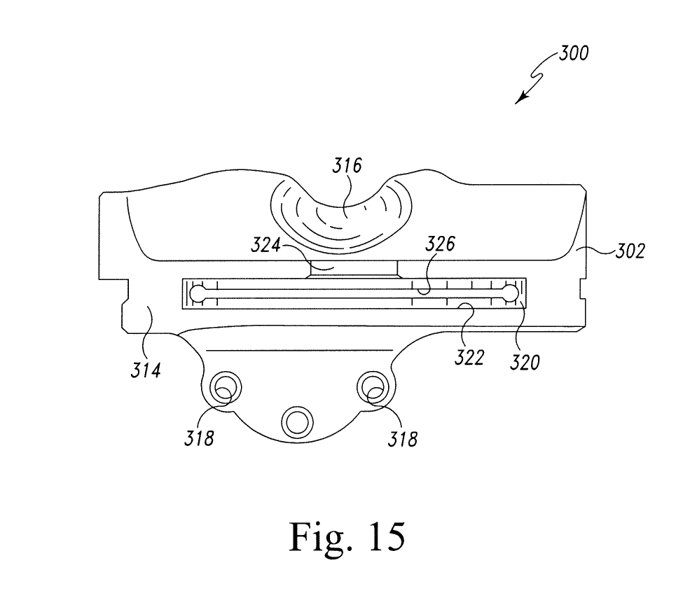

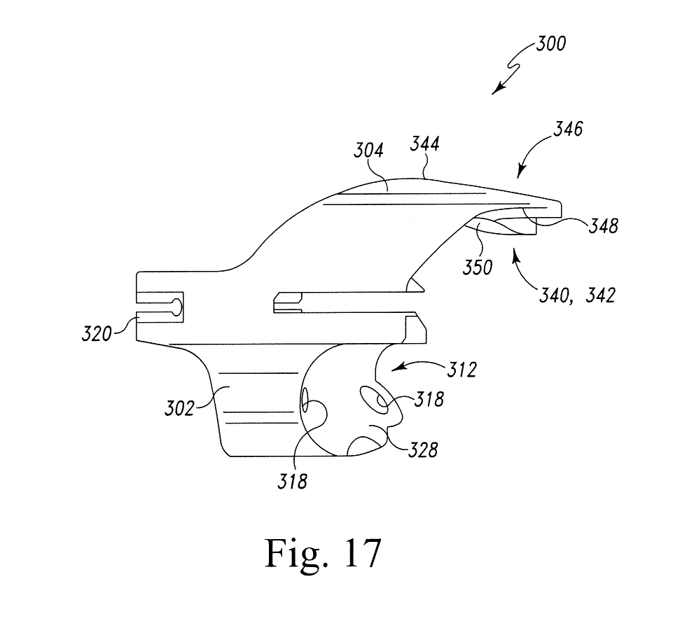

Referring now to FIGS. 15-19, in another embodiment, the customized patient-specific orthopaedic surgical instrument may be embodied as a tibial cutting block 300. The cutting block 300 is configured to be coupled to a tibia of a patient. The cutting block 300 includes a body 302 configured to be coupled to the anterior side of the patient's tibia and two arms or tabs 304, 306, which extend away from the body 302 in a posteriorly direction. The tabs 304, 306 are configured to wrap over a proximal end of the tibia as discussed in more detail below. The cutting block 300 may be formed from any suitable material. For example, the cutting block 300 may be formed from a material such as a plastic or resin material. In one particular embodiment, the cutting block 300 is formed from Vero resin using a rapid prototype fabrication process. However, the cutting block 300 may be formed from other materials in other embodiments. For example, in another particular embodiment, the cutting block 300 is formed from a polyimide thermoplastic resin, such as a Ultem resin, which is commercially available from Saudi Basic Industries Corporation Innovative Plastics of Riyhadh, Saudi Arabia.

The body 302 includes a bone-contacting or bone-facing surface 312 and an outer surface 314 opposite the bone-facing surface 312. The outer surface 314 includes a depression or recessed area 316, which provides an indication to a surgeon where to apply pressure to the body 302 when coupling the cutting block 300 to the patient's tibia. Additionally, a number of guide pin holes or passageways 318 are defined through the body 302 and have a diameter sized to receive respective guide pins to secure the block 300 to the patient's tibia. In some embodiments, one or more of the guide pin holes 318 may be oblique or otherwise angled with respect to the remaining guide pin holes 318 to further secure the block 300 to the patient's bone.

The body 302 includes a modular cutting guide 320. That is, the body 302 includes a cutting guide receiver slot 322 in which the cutting guide 320 is received. A latch 324 or other locking device secures the cutting guide 320 in place in the cutting guide receiver slot 322. As such, one of a number of different cutting guides 320 having a cutting guide slot 326 defined in various offset positions may be coupled to the body 302 to allow a surgeon to selectively determine the amount of bone of the patient's bone is removed during the bone cutting procedure. For example, a cutting guide 320 having a cutting guide slot 326 offset by +2 millimeters, with respect to a neutral reference cutting guide 320, may be used if the surgeon desires to remove a greater amount of the patient's bone. The cutting guide 320 may be formed from the same material as the body 302 or from a different material. In one particular embodiment, the cutting guide 320 is formed form a metallic material such as stainless steel. It should be appreciated that the cutting block 300 may be embodied without a modular cutting guide 320. That is, the cutting block 300 may be embodied with a fixed cutting guide 320 that is overmolded into the polymer body 302

The bone-facing surface 312 of the body 302 includes a negative contour 328 configured to receive a portion of the anterior side of the patient's tibia having a corresponding contour. As discussed above, the customized patient-specific negative contour 328 of the bone-contacting surface 312 allows the positioning of the cutting block 300 on the patient's tibia in a unique pre-determined location and orientation.

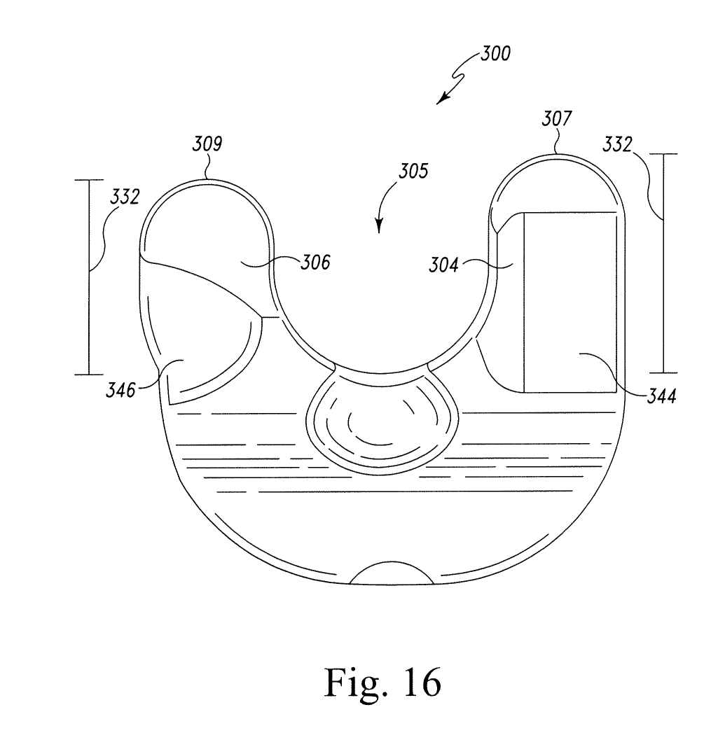

As discussed above, the arms or tabs 304, 306 extend posteriorly from the body 302 to define a U-shaped opening 305 therebetween. The tabs 304, 306 may extend from the body 302 the same distance or a different distance. For example, as shown in FIG. 16, the tab 304 extends from the body 302 a distance 330 and the tab 306 extends from the body 302 a distance 332, which is greater than the distance 330. The tabs 304, 306 taper in the anterior-posterior direction. That is, the thickness of the tabs 304, 306 at an anterior end of the tabs 304, 306 is greater than the thickness of the tabs 304, 306 at a respective posterior end 307, 309. The tapering of the tabs 304, 306 allow the tabs 304, 306 to be inserted within the joint gap defined between the patient's femur and tibia.

The tabs 304, 306 include a bone-contacting or bone-facing surface 340, 342, respectively, and an outer surface 344, 346, respectively, opposite the bone-facing surface 340, 342. The bone-facing surface 340 of the tab 304 includes a negative contour 348 configured to receive a portion of the patient's proximal tibia having a respective corresponding contour. Similarly, the bone-facing surface 342 of the tab 306 includes a negative contour 350 configured to receive a portion of the patient's proximal tibia having a respective corresponding contour.

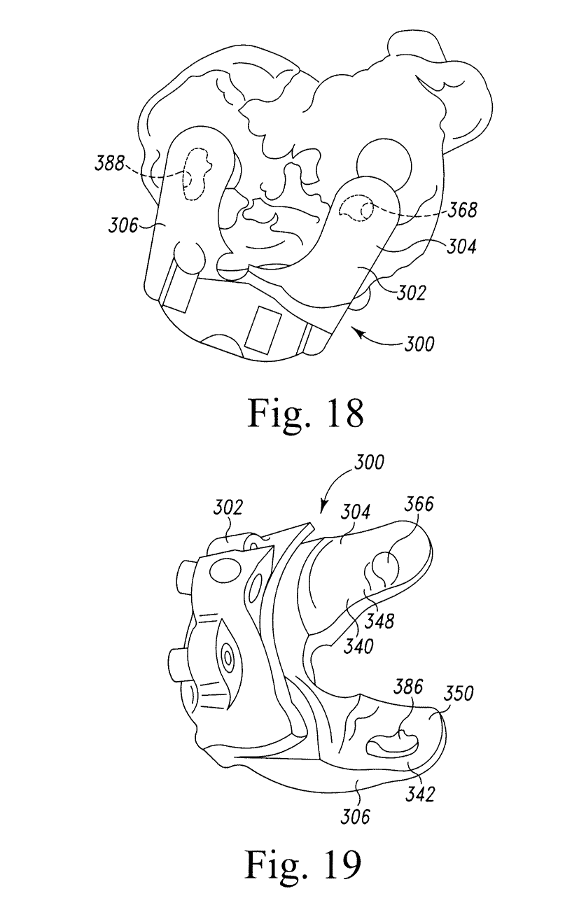

As can be seen in FIGS. 18 and 19, each of the negative contours 348, 350 of the tabs 304, 306, respectively includes a protrusion that corresponds to a cartilage void located on the proximal end of the patient's tibia. In particular, the negative contour 348 of the tab 304 includes a protrusion 366 that is sized, shaped, and positioned to be snuggly received into a cartilage defect 368 located on the patient's proximal tibia. The size, shape, and position of the cartilage defect 368 was pre-operatively determined as described above and the corresponding data was utilized in the fabrication of the cutting block 300. Similarly, the negative contour 350 of the tab 306 includes a protrusion 386 that is sized, shaped, and positioned to be snuggly received into a cartilage defect 388 located on the patient's proximal tibia. Like the cartilage defect 368, the size, shape, and position of the cartilage defect 388 was pre-operatively determined as described above and the corresponding data was utilized in the fabrication of the cutting block 300.

In addition, in some embodiments, the negative contours 328, 348, 350 of the bone-contacting surfaces 312, 340, 342 of the cutting block 300 may or may not match the remaining corresponding contour surface of the patient's bone. That is, as discussed above, the negative contours 328, 348, 350 may be scaled or otherwise resized (e.g., enlarged) to compensate for the patient's cartilage or lack thereof.

In use, the tibial cutting block 300 is coupled to the proximal end of the patient's tibia. Again, because the bone-contacting surfaces 312, 340, 342 of the cutting block 300 include the negative contours 328, 348, 350, the block 300 may be coupled to the patient's tibia in a pre-planned, unique position. When so coupled, the tabs 304, 306 wrap around the proximal end of the patient's tibia. Additionally, when the block 300 is coupled to the patient's tibia, a portion of the anterior side of the tibia is received in the negative contour 328 of the body 302 and a portion of the proximal side of the patient's tibia is received in the negative contours 348, 350 of the tabs 304, 306. As such, the anterior and proximal surfaces of the patient tibia are referenced by the tibial cutting block 300. Moreover, when the proximal end of the patient's tibia is received into the negative contours 348, 350 of the tabs 304, 306, the protrusion 366 formed on the tab 304 is snuggly received into the cartilage defect 368, and the protrusion 386 formed on the tab 306 is snuggly received into the cartilage defect 388, thereby facilitating placement of the cutting block 300 in the desired, pre-operatively determined location on the patient's tibia.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the apparatus, system, and method described herein. It will be noted that alternative embodiments of the apparatus, system, and method of the present disclosure may not include all of the features described yet still benefit from at least some of the advantages of such features. Those of ordinary skill in the art may readily devise their own implementations of the apparatus, system, and method that incorporate one or more of the features of the present invention and fall within the spirit and scope of the present disclosure as defined by the appended claims.

What is claimed is:

1. A method for designing a customized patient-specific bone cutting block for use in an orthopaedic surgical procedure to perform a bone cut on a patient's bone, the method comprising:
determining cartilage defect data indicative of the location, size, and shape of a cartilage defect present on an end of the patient's bone;
generating a reference contour based on the cartilage defect data; and
creating a customized patient-specific negative contour of the customized patient-specific bone cutting block using the reference contour.

2. The method of claim 1, wherein generating a reference contour comprises generating a reference contour based on a surface contour of a three-dimensional model of the patient's bone.

3. The method of claim 1, wherein determining cartilage defect data comprises determining cartilage defect data indicative of the location, size, and shape of a cartilage void.

4. The method of claim 3, wherein creating the customized patient-specific negative contour comprises creating a customized patient-specific negative contour that includes a protrusion that is sized, shaped, and positioned to be received into the cartilage void when the customized patient-specific cutting block is secured to the patient's bone.

5. The method of claim 1, wherein determining cartilage defect data comprises determining cartilage defect data indicative of the location, size, and shape of a cartilage protrusion.

6. The method of claim 5, wherein creating the customized patient-specific negative contour comprises creating a customized patient-specific negative contour that includes a void that is sized, shaped, and positioned to receive the cartilage protrusion when the customized patient-specific cutting block is secured to the patient's bone.

7. The method of claim 1, wherein:
determining cartilage defect data comprises determining cartilage defect data present on the distal end of the patient's femur, and
creating the customized patient-specific negative contour comprises creating a customized patient-specific negative contour of a customized patient-specific femoral cutting block.

8. The method of claim 1, wherein:
determining cartilage defect data comprises determining cartilage defect data present on the proximal end of the patient's tibia, and
creating the customized patient-specific negative contour comprises creating a customized patient-specific negative contour of a customized patient-specific tibial cutting block.

9. A method for designing a customized patient-specific bone cutting block for use in an orthopaedic surgical procedure to perform a bone cut on a patient's bone, the method comprising:
determining cartilage defect data indicative of the location and size of a cartilage void present on an end of the patient's bone;
generating a reference contour based on the cartilage defect data; and
creating a customized patient-specific negative contour of the customized patient-specific bone cutting block using the reference contour, the customized patient-specific negative contour comprising a protrusion that is sized and positioned to be received into the cartilage void when the customized patient-specific cutting block is secured to the patient's bone.

10. The method of claim 9, wherein generating a reference contour comprises generating a reference contour based on a surface contour of a three-dimensional model of the patient's bone.

11. The method of claim 9, wherein determining cartilage defect data further comprises determining cartilage defect data indicative of the shape of the cartilage void.

12. The method of claim 9, wherein determining cartilage defect data further comprises determining cartilage defect data indicative of the location, size, and shape of a cartilage protrusion present on the relevant end of the patient's bone.

13. The method of claim 12, wherein creating the customized patient-specific negative contour further comprises creating a customized patient-specific negative contour that includes a void that is sized, shaped, and positioned to receive the cartilage protrusion when the customized patient-specific cutting block is secured to the patient's bone.

14. The method of claim 9, wherein:
determining cartilage defect data comprises determining cartilage defect data present on the distal end of the patient's femur, and
creating the customized patient-specific negative contour comprises creating a customized patient-specific negative contour of a customized patient-specific femoral cutting block.

15. The method of claim 9, wherein:
determining cartilage defect data comprises determining cartilage defect data present on the proximal end of the patient's tibia, and
creating the customized patient-specific negative contour comprises creating a customized patient-specific negative contour of a customized patient-specific tibial cutting block.

16. A customized patient-specific cutting block, comprising:
a bone-facing surface including a customized patient-specific negative contour configured to receive a portion of a patient's bone having a corresponding positive contour, the customized patient-specific negative contour comprising a protrusion that is sized and positioned to be received into a cartilage void of corresponding size and position when the customized patient-specific cutting block is secured to the patient's bone.

17. The customized patient-specific cutting block of claim 16, wherein the bone-facing surface comprises a customized patient-specific negative contour configured to receive a portion of a patient's femur having a corresponding positive contour.

18. The customized patient-specific cutting block of claim 16, wherein the bone-facing surface comprises a customized patient-specific negative contour configured to receive a portion of a patient's tibia having a corresponding positive contour.
